(19) United States
(12) Patent Application Publication
(10) Pub. No.: US 2024/0197208 A1
(43) Pub. Date: Jun. 20, 2024

(54) METHOD FOR ENCODING MOTION SENSOR DATA, AND ASSOCIATED DEVICE AND SYSTEM

(71) Applicant: Zhor Tech, Nancy (FR)

(72) Inventors: Mathieu CHRETIEN, Nancy (FR); Brieuc BERRUET, Nancy (FR); Radouane OUDRHIRI, Nancy (FR); Karim OUMNIA, Nancy (FR)

(21) Appl. No.: 18/554,572

(22) PCT Filed: Apr. 14, 2022
(86) PCT No.: PCT/FR2022/050712

§ 371 Date: Feb. 9, 2024

(30) Foreign Application Priority Data
Apr. 14, 2021 (FR) FR2103875
Apr. 14, 2021 (FR) FR2103876

(51) Int. Cl. A61B 5/11; A61B 5/00

(52) U.S. Cl. CPC A61B 5/1126; A61B 5/6807; A61B 5/7267; A61B 2562/0219

(57) ABSTRACT

The invention relates to a device (20) for encoding data generated by one or more motion sensors (21), a system (5) for determining the displacement of a mobile entity, in particular a user, and a method (1) for encoding data generated by one or more motion sensors (21) coupled to a mobile entity such as a shoe, comprising: acquiring (200) data: computing (300) a plurality of descriptors over a time window (31); coding each of the descriptors on at most eight bits, said bits associated with a time window (31) being representative of a motion phenomenon of the mobile entity such as the shoe: and generating (500) a data tensor comprising the bits obtained for each of the time windows (31), the data tensor comprises an encoding of a combination of motion phenomena representative of a movement of the mobile entity.

The invention focuses on the field of the study of the displacement and more particularly on the processing of raw data coming from movement sensors to facilitate the real-time study of the movements, position or more broadly activity of a moving entity. Particularly, the invention focuses on the field of the study of the gait and more particularly on the processing of raw data coming from inertial units to facilitate the real-time study of the movements, posture or more broadly activity of a person. The invention relates to a method for encoding data generated by one or several movement sensors preferably coupled to a moving entity such as a shoe. The invention further relates to a device for encoding and a system for encoding data generated by one or several movement sensors capable of implementing such a method.

### PRIOR ART

The study of the displacement finds applications in a large number of different fields. Particularly, the study of the displacement aims to understand how the movements performed by an entity when it moves are broken down. It finds for example application within the framework of the analysis of the human gait, which is a complex and cyclical process mainly aimed at supporting the vertical position and maintaining balance in static or dynamic conditions. Such a process requires the synergy of the muscles, the bones and the nervous system. Furthermore, the study of the movement or other applications, for example in displacement finds robotics, in order to allow the coordination of the limbs of a robot, or more generally to allow the analysis and representation of the displacements of an entity (e.g. object, individual) over time.

Many systems have been developed over the years to study the movements/displacements of an entity and for example human gait. These systems include for example the use of pressure sensors or inertial sensors positioned for example in the sole of a shoe, on the shoe or at the ankle. Particularly, several teams have focused on the use and the processing of the data from inertial sensors for the analysis of the human movement or more broadly the analysis of moving entities such as drones. These systems, based on one or several sensors, today provide classic “walking” parameter values indicating for example information on the number of steps thanks to calculations most often entirely carried out on external terminals.

Furthermore, these systems most often rely on pressure sensors or inertial sensors coupled to remote central calculation units in external devices with strong calculation capabilities. Thus, current systems for studying the movement or the gait generally include the transmission of high-frequency data from the embedded to the central calculation units. These systems must be capable of operating continuously without having to change/recharge the battery frequently while offering detailed analysis of the movement or gait of a user. However, in conventional systems, the real-time transmission of the raw data leads to high energy consumption while these systems must be able to operate autonomously over a long duration. For example, as part of a solution for monitoring the activity of a user by analyzing the data from the movement sensors, it was proposed to code the voltage data generated by the inertial units on 8 bits (US2007061105). However, such a solution reduces the accuracy of the transmitted voltage data which may have an impact on the overall accuracy of the solution. Alternatively, the transmission of exclusively preprocessed data limits the possibilities of evolution of the interpretations of the data measured by the movement sensors. Furthermore, there has been proposed a solution for recognizing a movement combining the analysis of data from a movement sensor with audio data (U.S. Ser. No. 10/978,093).

A study carried out in 2018 shows that of the 14 systems evaluated, the median of autonomy of the proposed systems is located less than 6 hours. This is clearly insufficient for consumer applications where a monitoring of the gait of an individual over at least one day is desired (“A survey on smart shoe insole systems”, Salma Saidani et al. 2018 10.1109/SMARTNETS.2018.8707391; ©2018 IEEE).

One solution to increase the autonomy of the systems for studying the gait of an individual is to increase the size of the battery. However, such a solution cannot be applied in all situations, in particular when a miniaturization of the monitoring devices is desired. For example, there have been proposed inertial units capable of operating for approximately 35 hours by sending the raw data to an external device capable of analyzing them (“Bluetooth Embedded Inertial Measurement Unit for Real-Time Data Collection for Gait Analysis”, Nimsiri Abhayasinghe et al. October 2013; Conference: Indoor Positioning and Indoor Navigation. Montbeliard (France)). However, such an inertial unit has dimensions of the order of 50 cm³ and a weight of the order of 50 grams. Such dimensions are in particular imposed by the use of a battery having sufficient capacity to continuously transmit the data to an external processing device.

Other solutions, already proposed by the Applicant (WO2019193301, WO2019077266, WO2020217037), are to allow at least partial calculation t the level of the movement measuring device so as not to transfer all of the measured data and thus reduce the consumption associated with the storage and/or transfer of data. However, such a solution presumes to have all the algorithms already integrated into the measuring device. However, in some cases, only part of the data processing algorithms has been integrated into the measuring device. There may also be a need to evolve the data processing methods.

Thus, there is a need for a method for encoding the data from a movement sensor allowing data storage and transfer that is less energy intensive at the measuring device while minimizing the loss of data.

### Technical Problem

The invention aims to overcome the drawbacks of the prior art. Particularly, the aim of the invention is to propose a method for encoding data generated by one or several movement sensors preferably coupled to a moving entity or a shoe, capable of generating/transmitting faithful information on the measured data and sufficient for the study of the movement of the moving entity equipped with said movement sensors or of the gait of the individual wearing said movement sensors. Such a method allows systems to operate for longer durations without having to change/recharge the battery, while offering a detailed analysis of the movement and particularly of the gait of a user. Furthermore, such a method makes it possible to determine the movement of a moving entity or the gait of a user in real time.

The invention further aims to propose a device for encoding data and a system for determining the movement of a moving entity or the gait of a user integrating such a device, said system having improved autonomy while being capable of studying the movement of the moving entity or the gait of the user in real time.

### BRIEF DESCRIPTION OF THE INVENTION

The invention aims to overcome these drawbacks. The following presents a simplified summary of selected aspects, embodiments and examples of the present invention for the purpose of providing a basic understanding of the invention. However, this summary does not constitute an exhaustive overview of all aspects, embodiments and examples of the invention. Its sole aim is to present selected aspects, embodiments and examples of the invention in a concise form as an introduction to the more detailed description of the aspects, embodiments and examples of the invention which follow the summary.

To this end, the invention relates to a method for encoding data generated by one or several movement sensors coupled to a moving entity, for the identification of a position or movement of the moving entity on a time sequence, said method, executed by one or several processors, including:
a step of acquiring data generated by the movement sensor (s) coupled to the moving entity, said data including variable values in the form of time series, preferably said variable values include acceleration and angular speed values;
a step of calculating, from the data acquired over a time window, a plurality of descriptors, said calculation step being repeated for several consecutive time windows, the consecutive time windows forming the time sequence; and
a step of transforming the plurality of descriptors of each of the consecutive time windows forming the time sequence, said transformation step including a generation, for each of the descriptors, of a data coded on at most eight bits, preferably on at most four bits;
said data coded on at most eight bits associated with the time sequence being representative of a position or movement of the moving entity on said time sequence.

Particularly, the invention relates to a method for encoding data generated by one or several movement sensors coupled to a shoe, for the identification of a position or movement of the shoe on a time sequence, said method, executed by one or several processors, including:
a step of acquiring data generated by the movement sensor (s) coupled to the shoe, said data including variable values in the form of time series, preferably said variable values include acceleration and angular speed values;
a step of calculating, from the data acquired over a time window, a plurality of descriptors, said calculation step being repeated for several consecutive time windows, the consecutive time windows forming the time sequence; and
a step of transforming the plurality of descriptors of each of the consecutive time windows forming the time sequence, said transformation step including a generation, for each of the descriptors, of a data coded on at most eight bits, preferably on at most four bits;
said data coded on at most eight bits associated with the time sequence being representative of a position or movement of the shoe on said time sequence.

The Applicant has developed an embeddable method capable of processing almost instantly (e.g. time less than 1 ms with a processor rate at 64 MHZ), preferably by processors coupled to the movement sensors, the raw data generated by the movement sensors. The movement sensors can be positioned at the level of a shoe (on the shoe, in a sole, etc.) which is worn by a user. This makes it possible to encode them in a format that could either be processed at the level of a processor coupled to the movement sensor, for example in an embedded device such as a bracelet or a telephone or a sole, or transmitted occasionally to an external terminal with reduced energy consumption and/or reduced storage memory.

The Applicant has particularly developed an encoding of data allowing better interpretation by machine learning of the movements. Without being bound by theory, this encoding can be considered as a phonemization of the time series derived from the movement sensors. Thus, the method will generate from a time series of data from a movement sensor, a set of “phonemes” capable of describing consecutive intervals of the time series. The combination or the particular assembly of these phonemes will make it possible to define particular positions, postures or movements.

Furthermore, such encoding also gives the possibility to an algorithm to process the encoded data in real time to translate it into predictions of displacements of a moving entity or of a gait of an individual (e.g. movement and/or posture).

The data thus encoded can then be processed or transmitted in the form of a data tensor. The method then includes a step of generating a data tensor including the data coded on at most eight bits obtained for several of the time windows, so that the data tensor includes an encoding representative of a position, posture or movement of the moving entity or of the shoe. Furthermore, the data tensor can include an encoding representative of a plurality of positions, postures or movements of the moving entity or of the shoe.

According to other optional characteristics of the method, the latter can optionally include one or several of the following characteristics, alone or in combination:
the one or several movement sensors as well as the one or several processors are integrated into an encoding device, preferably positioned in an accessory worn by a user such as a watch or a bracelet. When the moving entity is an object, the encoding device could for example be integrated to the electronic circuits controlling the displacement of the object or into a dedicated electronic device. Particularly, the one or several movement sensors as well as the one or several processors are integrated into an encoding device, preferably positioned in a sole. Such a device embedding a movement sensor and processors makes it possible to carry out part of the processing as close as possible to the generation of a raw movement data and therefore to accelerate the processing and reduce the energy cost.
the encoding device is positioned in a watch or a bracelet. Alternatively, the encoding device is positioned in a removable sole. The presence of the encoding device in a watch or a bracelet or a removable sole is possible thanks to the low consumption of the method and makes it possible to use the method on several accessories such as footwear.
the calculation step includes a preprocessing of the acquired data. It may be advantageous to modify the data, for example via filtering, in order to speed up the subsequent processing and in some cases reduce the associated energy consumption.
it further includes a step of generating a data tensor including the data coded on at most eight bits obtained for several of the time windows, so that the data tensor includes an encoding representative of a position or movement of the moving entity.
the descriptors are calculated from mathematical functions applied to the preprocessed or non-preprocessed data generated by the movement sensor (s) coupled to the moving entity such as the shoe, and said applied mathematical functions including for example a calculation of average, of statistical asymmetry, of the kurtosis coefficient, of comparisons of values of the extrema or the quartiles, of positions of the extrema in a time series, of comparisons of the edge values of the subsets of the movement and/or of a calculation of a data topology.
it includes a step of extracting a time sequence which can correspond to a movement unit, said extraction step implementing a learning model, preferably an unsupervised learning model. A prior extraction of a movement unit can improve the prediction accuracy of the method while reducing the associated energy consumption.
it includes a step of analyzing the data coded on at most eight bits, preferably of the data tensor, as a function of an activity data of the moving entity, said analysis step comprising a comparison between the data coded on at most eight bits or preferably the data tensor and a database comprising reference data for the given activity. Particularly, it includes a step of analyzing the data coded on at most eight bits, preferably the data tensor, as a function of an activity data of the user, said analysis step comprising a comparison between the data coded on at most eight bits or preferably the data tensor and a database comprising reference data for the given activity. This advantageously makes it possible to determine which data are associated with the movements relating to an activity and which data correspond to risky or unintentional movements. The given activity can for example correspond to a displacement protocol, an operating protocol, a sports activity but also a relaxing or professional activity.
the time windows of two consecutive time sequences have different durations. Thus, the analysis can adapt to different types of displacements, different displacement cycle temporalities and does not require long segmentation of the data. Particularly, the analysis can adapt to different types of walks, different walking cycle temporalities and does not require a long segmentation of the data.
during the transformation step, at least four descriptors are used to each generate a data coded on at least one bit, more preferably on at least two bits.
during the transformation step, several descriptors are transformed into a single data of at most eight bits, preferably at most three bits. In some cases, “composite” descriptors formed from several descriptors may be the only ones needed. Particularly, during the transformation step, at least eight descriptors are used, to each generate a data coded on at least one bit, more preferably at least two bits.
one or several data coded on at most eight bits, preferably at most three bits, are calculated from values of descriptors calculated from data acquired over consecutive time windows. In some cases, a “composite” descriptor formed from several descriptors coming from different time windows can improve the performance.
during the transformation step, between four and twenty descriptors are used to each generate a data coded on a number of bits selected from: one, two or three. Such values make it possible to obtain better performance in terms of data retention, storage volume and power consumption.
the moving entity is a shoe, and the acquisition step is carried out while the one or several movement sensors as well as the one or several processors are integrated to an encoding device, preferably positioned in a sole.

According to a second object, the invention relates to a method for determining a displacement of a moving entity, from data likely to have been encoded according to an encoding method according to the invention, said determination method, executed by one or several processors, including:
a step of loading one or several data tensors including an encoding representative of a position or movement of the moving entity, preferably said one or several data tensors including a plurality of descriptors for each of the consecutive time windows forming a time sequence, each of the descriptors corresponding to a data coded on at most eight bits; and
a step of processing the data tensor (s) by a machine learning model trained so as to identify a position or movement of the moving entity.

Particularly, the invention relates to a method for determining the gait of a user of a shoe, from data likely to have been encoded according to an encoding method according to the invention, said determination method, executed by one or several processors, including:
a step of loading one or several data tensors including an encoding representative of a position or movement of the user's shoe, preferably y said one or several data tensors including a plurality of descriptors for each of the consecutive time windows forming a time sequence, each of the descriptors corresponding to a data coded on at most eight bits; and
a step of processing the data tensor (s) by a machine learning model trained so as to identify a posture or movement of the user.

According to other optional characteristics of the method, the latter can optionally include one or several of the following characteristics, alone or in combination:
the displacement of the moving entity includes one or several movements and/or positions of the moving entity. Particularly, the displacement of the moving entity can correspond to the user's gait. The user's gait preferably includes one or several movements and/or postures of the user.
the step of processing the data tensor (s) by a machine learning model includes the identification of classes of events from one or several data tensors, preferably one class of movement per time sequence.
it includes a step of selecting, preferably by the trained machine learning model, a position or movement from a comparison of the positions or movements identified from contiguous time windows or contiguous time sequences. Particularly, it includes a step of selecting, preferably by the trained machine learning model, a posture or movement of the user from a comparison of the postures or movements of the user identified at from contiguous time windows or contiguous time sequences.
it includes the identification, by the trained learning model, of a position or movement for each of several contiguous time sequences, the method then including a selection of the position or movement most represented within the contiguous time sequences. Particularly, it includes the identification, by the trained learning model, of a posture or movement for each of several contiguous time sequences, the method then including a selection of the posture or movement most represented within the contiguous time sequences. Such a step makes it possible to reduce the risk of false positives by taking advantage of the real-time recognition of time series and of a multiple validation of the same position or posture or movement. For example, a movement can be selected from an analysis of at least three, preferably at least four and even more preferably at least five contiguous time sequences.
it includes a step of comparing the positions and/or movements identified for a given time window or time sequence from data from different sensors so as to identify a position or movement of the moving entity. Particularly, it includes a step of comparing postures and/or movements of the user identified for a given time window or time sequence from data from different sensors so as to identify a posture or movement of the user. For example, it can include, for a movement unit, a comparison of the movement identified for each of the arms, feet, or a comparison with a reference frame, so as to determine a position, posture or movement like those of a user coupled to the movement sensors.
the moving entity is a user of a shoe, the encoding representative of a position or movement of the moving entity is an encoding representative of a position or movement of the shoe of the user; and the step of processing the data tensor (s) makes it possible to identify a posture or movement of the user.

The invention further relates to a device for encoding data generated by one or several movement sensors coupled to a moving entity, for the identification of a position or movement of the moving entity on a time sequence, said encoding device including one or several processors, said one or several processors being configured to:
acquire the data generated by one or several movement sensors coupled to the moving entity, said data including variable values in the form of time series, preferably acceleration and angular speed values;
calculate, from the data acquired over a time window, a plurality of descriptors, the calculation being repeated for several consecutive time windows, the consecutive time windows forming a time sequence;
transform the plurality of descriptors of each of the consecutive time windows forming the time sequence, said transformation including a generation for each of the descriptors of a data coded on at most eight bits, preferably at most three bits, said data coded on at most eight bits associated with a time window being representative of a movement or position of the moving entity on said time sequence.

Particularly, the invention further relates to a device for encoding data generated by one or several movement sensors coupled to a shoe, for the identification of a position or movement of the shoe on a time sequence, said encoding device including one or several processors, said one or several processors being configured to:
acquire the data generated by the movement sensor (s) coupled to the shoe, said data including variable values in the form of time series, preferably acceleration and angular speed values;
calculate, from the data acquired over a time window, a plurality of descriptors, the calculation being repeated for several consecutive time windows, the consecutive time windows forming a time sequence;
transform the plurality of descriptors of each of the consecutive time windows forming the time sequence, said transformation including a generation for each of the descriptors of a data coded on at most eight bits, preferably at most three bits, said data coded on at most eight bits associated with a time window being representative of a movement of the shoe on said time sequence.

Furthermore, the encoding device can include said one or several movement sensors. Preferably, the one or several movement sensors include at least one accelerometer and at least one gyroscope. Furthermore, the processor (s) can be configured to generate a data tensor including the data coded on at most eight bits obtained for several of the time windows, such that the data tensor includes an encoding representative of a position or movement of the moving entity. For example, the processor (s) can be configured to generate a data tensor including the data coded on at most eight bits obtained for several of the time windows, so that the data tensor includes an encoding representative of a position or movement of the shoe. The encoding device can be integrated to a removable or non-removable sole.

The invention further relates to a system for determining a position or movement of a moving entity, said determination system including at least one encoding device according to the invention and an external terminal configured to receive the generated data tensor then to determine the displacement of a moving entity from the generated data tensor. Particularly, the invention further relates to a system for determining the gait of a user, said determination system including at least one encoding device according to the invention and an external terminal configured to receive the generated data tensor then to determine the gait of a user from the generated data tensor.

Other advantages and characteristics of the invention will appear upon reading the following description given by way of illustrative and non-limiting example, with reference to the appended figures:

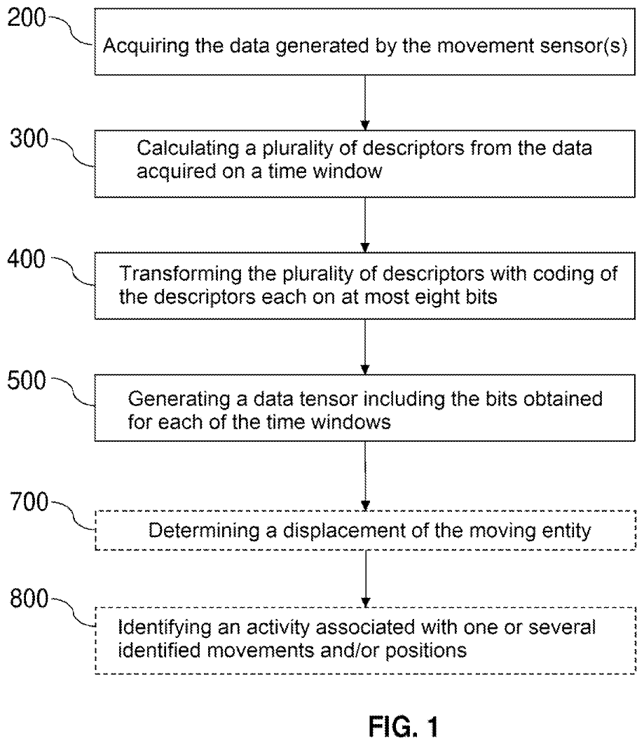

FIG. 1 represents a diagram of a method according to one embodiment of the invention. The dotted steps are optional.

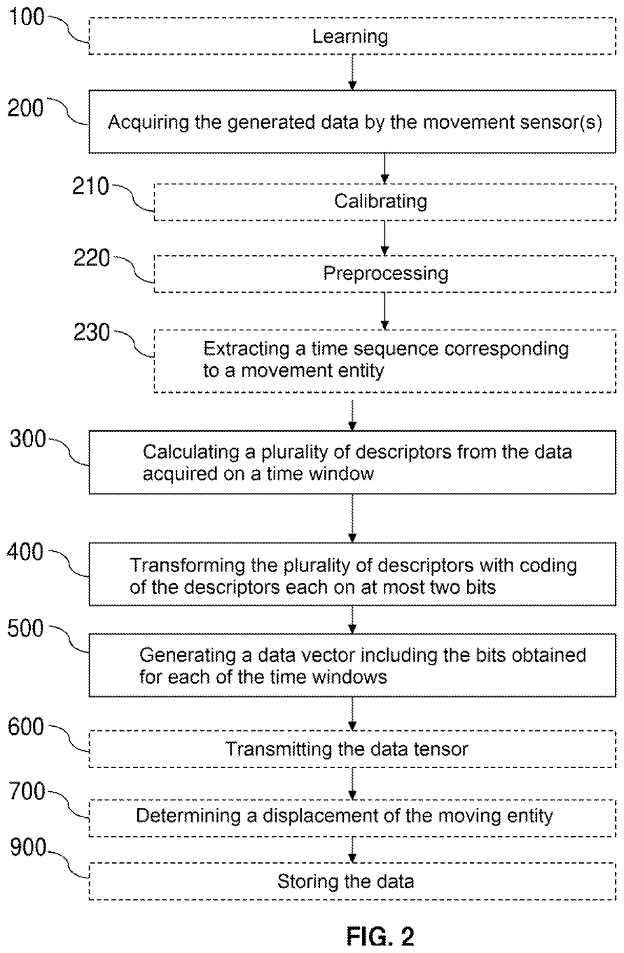

FIG. 2 represents a diagram of a method according to another embodiment of the invention. The dotted steps are optional.

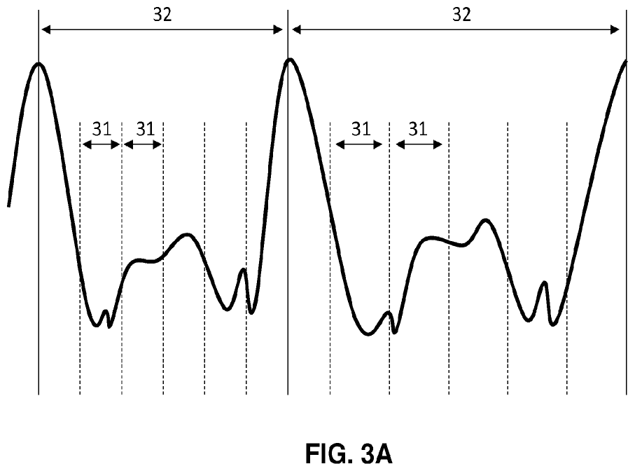

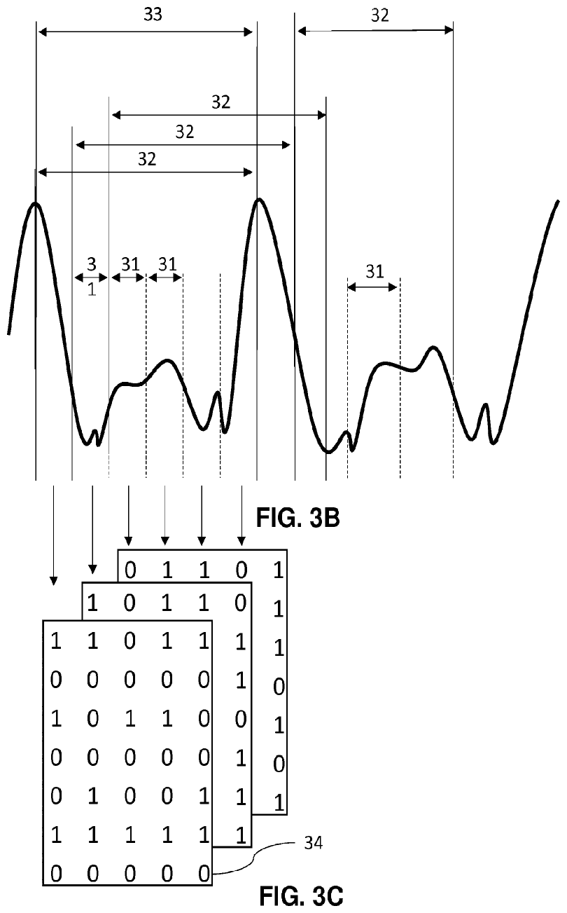

FIGS. 3A and 3B each represent graph showing generated data divided into a time sequence subdivided into time windows, by a movement sensor along the axis X. FIG. 3C corresponds to second-order data tensors and tensors derived respectively from time window data and time sequence data.

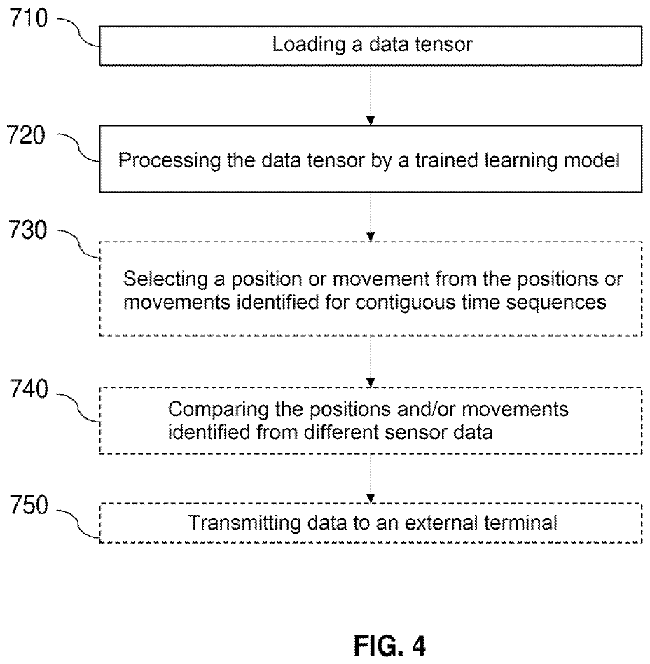

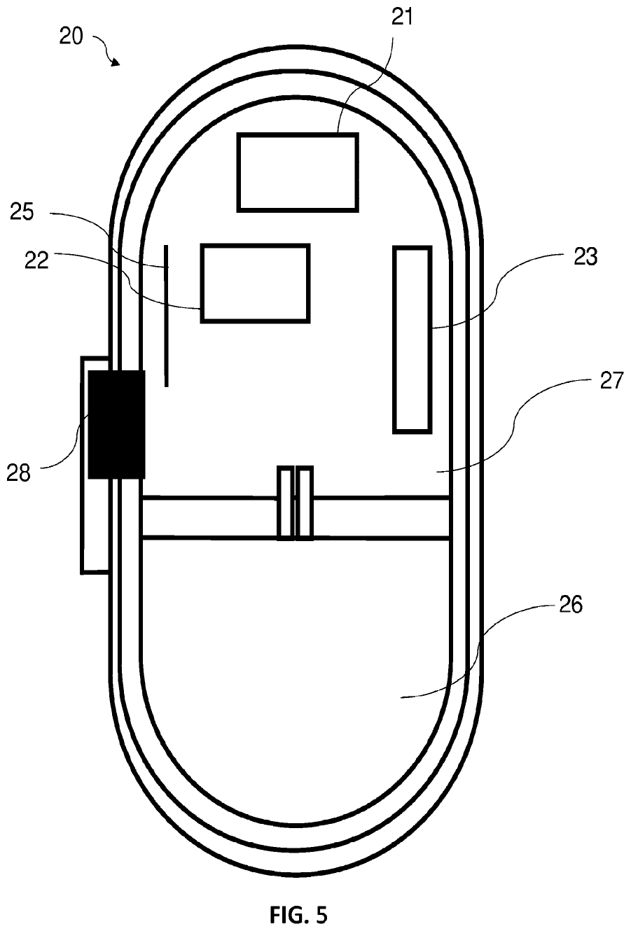

FIG. 4 represents a diagram of a method for determining the displacement of a moving entity or the gait of a user according to one embodiment of the invention. The dotted steps are optional.

FIG. 5 represents a diagram of an open encoding device seen from above comprising in particular an electronic card, an energy source, a connector and a communication means.

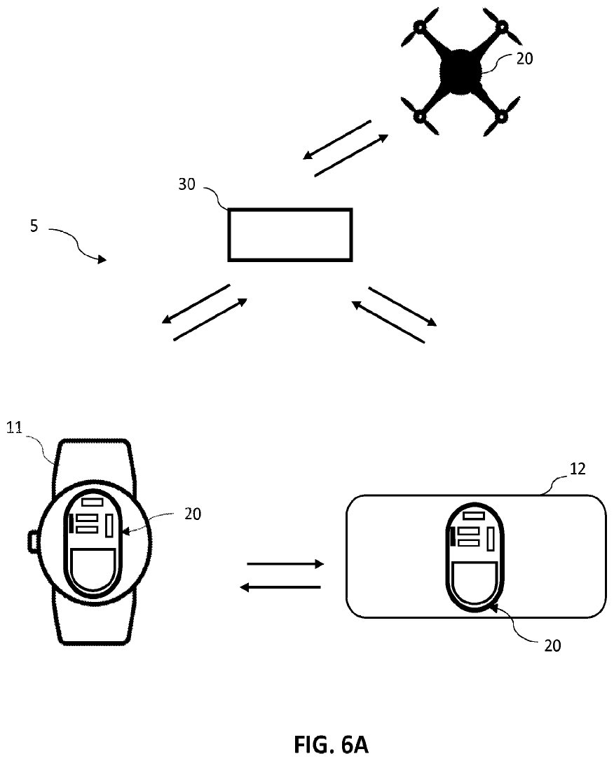

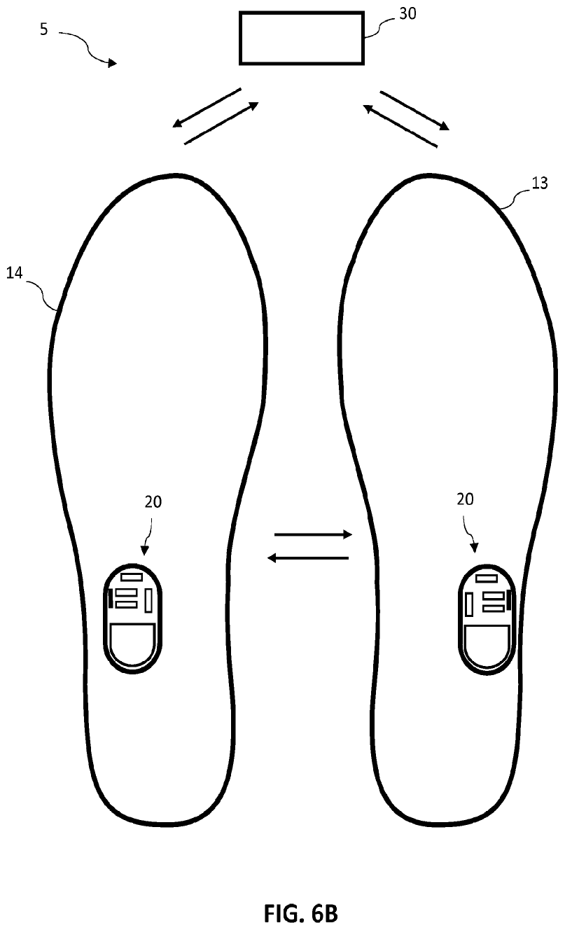

FIGS. 6A and 6B represent a diagram of a system for determining a displacement of a moving entity according to the invention. Particularly, FIG. 6B represents a diagram of a system for determining the gait of a user according to the invention.

Aspects of the present invention are described with reference to flowcharts and/or functional diagrams of methods, apparatuses (systems) and computer program products according to embodiments of the invention.

In the figures, flowcharts and functional diagrams illustrate the architecture, functionality and operation of possible implementations of systems, methods and computer program products according to various embodiments of the present invention. In this regard, each block in the flowcharts or block diagrams may represent a system, a device, a module or a code, which comprises one or several executable instructions to implement the specified logic function (s). In some implementations, the functions associated with the blocks may appear in a different order than the one indicated in the figures. For example, two blocks shown successively can in fact be executed substantially simultaneously, or the blocks can sometimes be executed in the reverse order, depending on the involved functionality. Each block of the flow diagrams and/or flowchart, and combinations of blocks in the flow diagrams and/or flowchart, can be implemented by special hardware systems that execute the specified functions or acts or perform combinations hardware and computer instructions.

### DESCRIPTION OF THE INVENTION

In the remainder of the description, the “displacement” within the meaning of the invention corresponds to the position, movements, locomotion and balance of the moving entity. The balance corresponds in particular to the stability of the body relative to the center of gravity of the moving entity. However, it can integrate both static balance and dynamic balance. The “gait” within the meaning of the invention preferably corresponds to the posture, movements, locomotion, and balance of the user. The balance corresponds in particular to the postural balance linked to the stability of the body and more particularly to the stability of the center of gravity of a user. However, it can integrate both static balance and dynamic balance.

The “determination of a displacement” corresponds, within the meaning of the invention, to the assignment of one or several values, for example a score, a classification or a note to a trajectory or to the movement of the moving entity, and as presented in connection with one embodiment, of an arm of a user. This determination of the displacement also makes it possible to obtain one or several numeric or alphanumeric values of biomechanical parameters representative of the displacement as a function of the considered moving entity. The “determination of the gait” corresponds, within the meaning of the invention, to the assignment of one or several values, for example a score, a classification or a note to a trajectory or to the displacement of foot of a user. This determination of the gait also makes it possible to obtain one or several numeric or alphanumeric values of biomechanical parameters representative of the gait. A solution (e.g. method, device, system, etc.) according to the invention could determine a position, posture or movement of the moving entity. In the present application and for the entire description, preferably, the invention makes it possible to determine a movement of a moving entity indeed a succession of positions makes it possible to deduce a movement. As part of the study of a user's gait, the invention can be used to determine a posture or a gait of a user and preferably a gait of a user.

The term “activity” within the meaning of the invention can correspond to an action carried out by the moving entity or the user over an extended period. This can for example correspond to a particular type of displacement, related by way of non-limiting examples to a sports activity such as football, cycling or basketball, but also to a sedentary activity such as an office activity or any other activity such as a recreational walk or a hike.

By “moving entity” it is meant any entity capable of moving or configured to move autonomously or non-autonomously. A moving entity can for example correspond to a living being, a vehicle, a robotic machine, a shoe or a drone.

The term “descriptor” within the meaning of the invention can preferably correspond to a value obtained via a mathematical transformation the data acquired from the movement sensors. A mathematical transformation could for example correspond to the calculation of an average, a median, a variance, statistical asymmetry, of the kurtosis coefficient, extrema values, quartile values, comparisons of values of the extrema or quartiles, positions of the extrema in a time series, and comparisons of the edge values of the subsets of the movement. For example, for a window, an edge value corresponds to the initial and final value of the window.

The expression “movement phoneme” within the meaning of the invention can correspond to a set of data calculated from several descriptors generated over a given time window. These data advantageously take the form within the framework of the invention of data encoded on at most eight bits. Thus, the movement phoneme corresponds to several values encoded on at most eight bits describing at least partly a movement over the time window studied. These movement phonemes can thus correspond to data calculated from several descriptors describing a sub-part of a movement called micromovement, that is to say a movement that can for example take place over less than 500 ms.

The expression “data tensor” within the meaning of the invention can correspond to an object or to a data structure, such as a one-dimensional table, a two-dimensional matrix or a cube including data relating to descriptor values transformed into values coded in at most eight bits, preferably in at most two bits. The representation tensor in form is a representation of the data allowing for example the recourse to matrix calculation operations, optimized in terms of calculation and memory complexity. Furthermore, the data tensors, when they correspond to the values of a sensor for a time window can form a third-order data tensor and can be processed jointly with data tensors for other sensors and data tensors for other time windows so as to form a list of third-order data tensors.

By “bio-mechanical parameter” it is meant within the meaning of the invention a characteristic of the gait of a moving entity when it corresponds to a human being or a robot whose displacements require a particular coordination, for example the gait of the user.

The term “accessory” corresponds to any item intended to be worn by a moving entity. An accessory according to the invention can take the form of a textile item or more generally of a garment. The accessory can also be adapted to be fixed to or integrated to a garment or take the form of a bracelet. The accessory can also be a removable modular element adaptable to a ski, walking sticks, a shoe (for example a cleat for cycling), a prosthesis, etc.

By “sole” it is meant an object making it possible to separate the user's foot from the ground. A shoe can include an upper sole layer in direct contact with the user's foot and a lower sole layer in direct contact with the ground or more generally the external environment. A shoe can also include a removable insole.

By “removable” it is meant the ability to be detached, removed or dismounted easily without having to destroy the fixing means either because there are no fixing means or because the fixing means are easily and quickly dismountable (e.g. notch, screw, tab, lug, clips). For example, by “removable” it should be understood that the object is not fixed by welding or by another means not intended to allow detaching the object.

By “model” or “rule” or “algorithm”, it should be understood within the meaning of the invention, a finite sequence of operations or instructions making it possible to calculate a value by means of a classification or partitioning of the data within previously defined groups Y and to assign a score or to prioritize one or several data within a classification. The implementation of this finite sequence of operations makes it possible for example to assign a label Y to an observation described by a set of characteristics or parameters X for example thanks to the implementation of a function f, capable of reproducing Y having observed X.

Y=f(X)+e

where e symbolizes noise or measurement error.

By “supervised learning method” it is meant within the meaning of the invention a method making it possible to define a function f from a base of n labeled observations (X_{1 . . . n}, Y_{1 . . . n}) where Y=f(X)+e.

By “unsupervised learning method” it is meant a method aimed at prioritizing the data or dividing a set of data into different homogeneous groups, the groups of homogeneous data sharing common characteristics and this without the observations being labeled.

By “substantially constant” it is meant a value varying by less than 15% relative to the compared value, preferably by less than 10%, even more preferably by less than 5%.

By “process”, “calculate”, “determine”, “display”, “transform”, “extract”, “compare” or more broadly “executable operation” it is meant within the meaning of the invention, an action performed by a device or a processor unless the context indicates otherwise. In this regard, the operations relate to actions and/or processes of a data processing system, for example a computer system or an electronic computing device, which manipulates and transforms the data represented as physical (electronic) quantities in the memories of the computer system or other information storage, transmission or display devices. These operations can be based on applications or software.

The terms or expressions “application”, “software”, “program code”, and “executable code” mean any expression, code or notation, of a set of instructions intended to cause data processing to directly or indirectly perform a particular function (e.g. after a conversion operation to another code). Examples of program code can include, but are not limited to, a subroutine, a function, an executable application, a source code, an object code, a library, and/or any other sequence of instructions designed for the execution on a computer system.

By “processor” it is meant within the meaning of the invention at least one hardware circuit configured to execute operations according to instructions contained in a code. The hardware circuit can be an integrated circuit. Examples of a processor comprise, but are not limited t central processing unit, a graphics processor, an application-specific integrated circuit (ASIC), and a programmable logic circuit.

By “computing device” it is meant any device comprising a processing unit or a processor, for example in the form of a microcontroller cooperating with a data memory, possibly a program memory, said memories are capable of being dissociated. The processing unit cooperates with said memories by means of an internal communication bus.

By “computing infrastructure” it is meant within the meaning of the invention a set of computing structures (i.e. computing devices) able to operate an application or an application chain. The computing structure can be a server and can for example be composed of a presentation server, a business server and a data server. Preferably, the computing structure is a server.

By “coupled” within the meaning of the invention it is meant directly or indirectly connected with one or several intermediate elements. Two elements can be mechanically, electrically coupled or linked by a communication channel.

In this description and even before, the same references are used to refer to the same elements.

The inventors have tested several methods and systems to study the displacement of a moving entity such as the gait of a user (e.g. position, posture and/or movement). They favored the systems integrating movement sensors, preferably inertial units, systems most likely to provide a rapid response and at a sustainable cost for industrialization. Indeed, existing methods are generally based on pressure sensors or on a plurality of sensors distributed in the shoe and/or sole or even on the ankle. Such a distribution of sensors leads to a reduction in the robustness of the system and/or an increase in its cost.

However, as has been mentioned, the vast majority of the systems for studying the displacement of a moving entity such as the gait of an individual suffer from the autonomy of the battery used, this being mainly caused by the transmission in real time of raw data generated by the movement sensors and particularly by inertial units. The alternative, namely dimensioning a battery capable of lasting several dozen hours, then concerns systems that could no longer be considered as portable or commonly used.

Faced with these inadequacies, the Applicant has developed an embeddable method capable of processing the raw data generated by the movement sensors almost instantly and encoding them in a format allowing transmission with reduced energy consumption and/or reduced storage memory. Furthermore, the method allows encoding such that the transmitted data, although requiring only a small amount of energy, retain a maximum of information and then allow a detailed analysis of the displacement of the moving entity or of the gait of the user by movement sensors.

Particularly, the Applicant has developed a solution for encoding data generated by one or several movement sensors. As has been mentioned, this encoding could be considered as based on a phonemization of the movement related to the displacement of a moving entity, particularly to the gait of an individual (e.g. position, posture and/or movement).

Such a solution could be applied to any type of moving entity and cannot be limited to the examples presented, in the remainder of the description, only for illustrative purposes. As already mentioned, the encoding solution could be applied to a moving entity when it corresponds to a vehicle, an aircraft or a robot, in particular an automated industrial robot (for example a robotic arm). Preferably, the encoding solution is particularly adapted to the embedded systems powered by batteries.

According to a first aspect, the invention relates to a method 1 for encoding data generated by one or several movement sensors. Particularly, such a method can be implemented for one or several movement sensors 21 coupled to a moving entity, such as a shoe, in particular a shoe worn by a user. It could in particular be implemented by one or several processors 22.

Thus, in order to illustrate the solution proposed by the Applicant, in the different examples or embodiments presented in the remainder of the description, the moving entity considered can correspond to a living being such as a human being (hereinafter referred to as “user”) and the encoding device will be presented as coupled, or integrated, to an object worn by the moving entity such as a garment, a watch, a bracelet or a mobile phone. The moving entity can also correspond to a motorized object capable of moving autonomously or by commands.

Particularly, such a method is implemented from data generated by one or several movement sensors 21 of an object such as a watch 11 or a sole 13, 14. Subsequently, the invention will be described in an implementation which could involve a watch or a bracelet but the invention can be implemented for any moving entity including one or several movement sensors or for any object worn by a moving entity. Some of the movement sensors 21 can be distributed in a watch 11, in soles 13, 14 or also be positioned on shoes so as to follow the movements of a user, such as his/her arm or the soles of his/her footwear. The movement sensors can also all be integrated into a first encoding device 20 associated with the watch 11 or with a first bracelet and a second encoding device 20 associated with a second bracelet positioned on the other arm of the user. The movement sensors can also all be integrated into a first encoding device 20 associated with a first shoe and a second encoding device 20 associated with a second shoe of the same pair. The invention can also be applied to data generated by inertial coupled to garments or connected textiles, arranged to detect movements of a wearer of said garment.

An encoding method 1 according to the invention will advantageously allow identification of a position or movement of a moving entity such as a shoe coupled to one or several movement sensors 21 on a time sequence ST, and in particular of the user's arm when the watch 11 and/or the bracelet 12 is (are) worn by the user but also when such a shoe is worn by a user.

As illustrated in FIG. 1, the encoding method 1 includes: a step 200 of acquiring data generated by the movement sensor (s) 21, a step 300 of calculating a plurality of descriptors from the data acquired over a time window 31, a step 400 of transforming the plurality of descriptors and a step 500 of generating a data tensor including the bits obtained for each of the time windows 31, so that the data tensor includes an encoding of a combination of several movement phonemes which is representative of a position or movement of the arm of the user or of a position or movement of the watch 11, the shoe or the bracelet 12 worn by the user.

Furthermore, a method according to the invention can include advantageous steps such as: a learning 100, a calibration 210 of a displacement of the moving entity such as the calibration of the user's gait, a preprocessing 220 of the data generated by the movement sensors and/or values calculated within the framework of the method, an extraction 230 of a time sequence corresponding to a movement unit, a transmission 600 of a data tensor, a determination 700 of a displacement of the moving entity (e.g. the user, a sole) or a storage 900 of the data. Particularly, the determination 700 of a displacement of the moving entity can correspond to the determination of the gait of a user.

In the remainder of the present description, the solution proposed by the Applicant will be described in one embodiment in which, the movement sensor (s) 21 include (s) at least one inertial unit of at least six axes and this or these movement sensors 21 is/are integrated into a watch 11 and/or a bracelet 12. It will also be described in one embodiment in which this or these movement sensors 21 can also be integrated into one or several soles 13, 14. The sole(s) involved in the present invention could or could not be removable. Preferably, the sole(s) involved in the present invention is/are removable.

However, the present invention in these different embodiments, preferred or not, could be applied in the case where the movement sensors are not integrated to a watch, a bracelet or a sole. It could be particularly applied when the movement sensors are positioned on a shoe, but also on the body of a user, for example integrated into a bracelet or into garment worn by the latter. Indeed, the movement data generated by one or several movement sensors positioned on the body of a user for example at the lower limbs or the spine could be encoded by an encoding method according to the invention and/or used to determine the displacement of a moving entity such as the user's gait.

The movement sensors could also be piezoelectric sensors, textile fibers or pressure sensors which, if positioned appropriately, can provide information on the movements of a moving entity, in particular of the user.

Within the framework of the following description, preferably, the one or several processors are positioned in a watch and/or a bracelet. Alternatively, the one or several processors could be positioned in a casing arranged to be fixed to a shoe or to a user. As will be described and illustrated, the one or several processors could be positioned in a sole, for example at an electronic casing.

Particularly, the one or several movement sensors as well as the one or several processors are integrated into an electronic casing, such as an encoding device 20, preferably positioned for example in an accessory of a user such as a garment, a watch, a bracelet or a sole.

As illustrated in FIG. 2, a method according to the invention can include a learning step 100.

A learning step 100 could include a step of collecting the training data then an aggregation and storage of these target data in a database. These training data may or may not be annotated and can be used to generate data tensors that can be used in a preferably distributed and parallelized training step.

Preferably, the learning step 100 could include the generation and selection of multiple learning models by cross-validation and selection of optimized hyper parameters. Finally, the learning step 100 can include a selection of the learning model providing the best classification performance, that is to say optimal and homogeneous performance between the classes.

Such a step can in particular include the definition of a plurality of activity reference values of a user. For example, during the repetition of movements, positions, postures and gaits performed by a user over a determined duration, activity reference values can be recorded and classified according to a plurality of values, including patterns, for example, of static or dynamic type.

A method according to the invention includes a step 200 of acquiring data generated by the movement sensor (s) 21, preferably coupled to a moving entity, in particular to a watch 11 and/or bracelet 12 worn by a user or a shoe. The acquisition, as can be understood from reading the present description, is preferably carried out for each of the accessories worn by the user and where appropriate to the watch 11 and/or bracelet 12. Particularly, it can be carried out for each of the two shoes and, where appropriate, of the two soles 13, 14 of the shoes. This step can correspond particularly within the framework of the invention to the acquisition of data generated by one or several movement sensors of an encoding device 20 or several encoding devices 20, for example each integrated to a watch 11, to a bracelet 12 and/or to a removable or non-removable sole. This acquisition could for example correspond to storing, preferably in a random access memory, the data generated by the sensors. This acquisition can also correspond to loading, into the memory, one or several processors intended to process them.

Particularly, the acquired data are raw data generated by the movement sensor (s) 21. Alternatively, the acquired data can also correspond to preprocessed data. As will be described below, the preprocessed acquired data can for example correspond to data which has been normalized, filtered, supplemented or even to data which have been merged for example by Kalman filtering. Preferably, the acquired data include variable values in the form of time series. These variable values could preferably correspond to acceleration and angular speed values, but they can for example also correspond to values generated by pressure sensors. They can possibly include magnetic field data or a fusion of these data. Thus, the data generated by the movement sensor (s) can also include magnetic field data. The acquired data could be coded on 8, 16, 32, 64, 128 bits.

This acquisition step can also include the acquisition of other data such as geolocation data, temperature data or pressure data.

Particularly, when the acquired data have been generated by movement sensors coupled to a moving entity, in particular positioned in a watch, a bracelet or a sole, these data will preferably correspond to acceleration and angular speed values of the moving entity, in particular of the watch, the bracelet or the sole.

Particularly, a method according to the invention can comprise a calibration step 210. As will be described subsequently, the movement sensors 21 can be associated with the moving entity, more particularly with an accessory such as a watch, a bracelet, a shoe and can be integrated into an encoding device 20.

A method according to the invention can also include a preprocessing 220 of the data generated by the movement sensors before or after their acquisition.

Particularly, this preprocessing step 220 can correspond to the preprocessing of the acceleration, angular speed and/or orientation values of a watch, a bracelet or a sole or of the pressure value. For example, it can particularly include at least one processing selected from: frequency filtering, suppression of gravity on the acceleration values, gravity suppression, suppression of noise on the acceleration values and suppression of the drift on the angular speed and/or orientation values of the watch, bracelet or sole.

As mentioned and illustrated in FIG. 2, a method according to the invention can include a step 230 of extracting a time sequence which can correspond to a movement unit.

The movement unit within the meaning of the invention can correspond to a sequence of one or several identical or different movements such as, by way of non-limiting examples, a flexion, a rotation, a translation or an extension. These movements can be defined as the period of time going from the initiation of a movement, for example of the arm of the user equipped with the watch 11 or the bracelet 12, to the next occurrence of the same event or the same movement with the same arm. The movement unit within the meaning of the invention can correspond to a walking cycle, a running cycle or a stair climbing cycle. These cycles can be defined as the period of time from the initial contact of a foot to the next occurrence of the same event with the same foot.

Within the framework of the invention, numerous partitioning methods can be used. Thus this step can for example include the implementation of conventional steps of studying the movement (e.g. the step), for example the Pan-Tompkins method or the detection of thresholds, maxima and/or minima.

The extraction 230 could advantageously implement a machine learning model, preferably an unsupervised machine learning model.

A method according to the invention also includes a step 300 of calculating a plurality of descriptors from the data acquired over a time window 31.

Advantageously, this calculation step can be repeated for several consecutive time windows. As will be described subsequently, the consecutive time windows 31 form a time sequence 32. This is in particular illustrated in FIGS. 3A and 3B.

FIGS. 3A and 3B represent data generated by a movement sensor, for example preprocessed or non-preprocessed acceleration data along the axis X. These data can be divided into time sequences 32, two in FIG. 3A, then subdivided into time windows 31.

As has been presented, a method according to the invention can include a step 230 of extracting a time sequence 32 which can correspond to a movement unit. Alternatively, a time sequence 32 could correspond to a sliding window as illustrated in FIG. 3B. Furthermore, as illustrated in FIG. 3B, preferably, the time sequences 32 can be overlapping.

The time window 31 does not necessarily correspond to a predetermined duration. Furthermore, preferably, it does not correspond to a predetermined duration. For example, the duration of the time window 31 can be defined once a movement unit or a type of movement has been identified.

Preferably, the method is configured to process two consecutive time sequences 32 not having the same duration, a time sequence 32 is divided into several time Preferably, windows 31 and in the same time sequence 32 all the time windows 31 will have the same size. However, when two consecutive time sequences 32 do not have the same duration then the time windows 31 of one of the two time sequences 32 will not have the same duration as the time windows of the other time sequence 32.

As illustrated in FIG. 3B, all the time windows 31 of a time sequence 32 can have an identical duration. Furthermore, the time windows 31 of two consecutive time sequences 32 may have different durations.

Thus, the duration of a time window can adapt to the duration of a movement unit. This makes it possible to consider displacements independently of their execution time.

For example, the duration of each of the time windows 31 of a time sequence 32 could correspond to a predetermined fraction of the duration of the time sequence 32.

Typically, the time windows 31 can have a duration greater than 200 ms, preferably greater than 400 ms, more preferably greater than 500 ms, and even more preferably greater than 600 ms. The time windows 31 can have a duration of less than 5 s, preferably less than 4 s, more preferably less than 3 s, and even more preferably less than 2 s. Particularly, the duration of a time t window 31 could for example be comprised between 25 and 1,000 ms while the duration of a time sequence 32 could for example be comprised between 100 and 5,000 ms. A time window 31 could for example represent between 1/20 and ¼ of a time sequence 32, preferably between 1/10 and ⅕ of a time sequence 32.

Advantageously, the descriptors are calculated from mathematical functions applied to the data generated by the movement sensor (s) 21, preprocessed or non-preprocessed. The mathematical functions preferably include calculations of statistical asymmetry, of kurtosis coefficient, of values of the extrema, of comparisons of the edge values of the subsets of the movement and/or of a calculation of data topology.

Furthermore, the implemented mathematical functions make it possible to calculate descriptors called “topological” descriptors making it possible to extract, measure and compare structural information hidden within complex data. In this case, the mathematical functions can be adapted to allow the calculation of the data topology via in particular the determination of a unidimensional or multidimensional persistence diagram (as presented in H. Edelsbrunner and J. Harer. Computational Topology: An Introduction. American Mathematical Society, 2009, or in H. Edelsbrunner, D. Letscher, and A. Zomorodian. Topological persistence and simplification. Disc. Compu. Geom., 2002), of the Reeb graph (J. Tierny, A. Gyulassy, E. Simon, and V. Pascucci. Loop surgery for volumetric meshes: Reeb graphs reduced to contour trees. IEEE Transactions on Visualization and Computer Graphics (Proc. of IEEE VIS), 2009.), or of the Morse-Smale complex (A. Gyulassy, P. T. Bremer, B. Hamann, and V. Pascucci. A practical approach to morse-smale complex computation: Scalability and generality. IEEE Transactions on Visualization and Computer Graphics (Proc. Of IEEE VIS), 2008). Of course, the calculation of these topological descriptors is not limited to the use of the mathematical functions described as illustrative examples, other mathematical functions can be used to determine other types of topological descriptors.

Preferably, the applied mathematical functions incorporate data coming from several movement sensors to generate a data coded on at most eight bits, preferably at most three bits, more preferably at most two bits. For example, in the case of an inertial unit generating acceleration data on three axes, a descriptor coded on at most three bits, preferably at most two bits, could have been generated from the values of the three axes.

Furthermore, the applied mathematical functions can advantageously incorporate data coming from an anterior time window to calculate a descriptor of a given window. Thus, the values of the descriptors can transcribe an evolution of the values generated by the movement sensors.

Preferably, the applied mathematical functions can also incorporate variables not coming from the movement sensors.

Preferably, the step 300 of calculating a plurality of descriptors will include the calculation of at least 4 descriptors, preferably at least six descriptors for each of the time windows 31.

A method according to the invention includes a step 400 of transforming the plurality of descriptors. This transformation step 400 of the plurality of descriptors advantageously includes a coding of descriptors, each on at most eight bits, preferably at most five bits, more preferably at most four bits, even more preferably at most three bits, for example each on at most two bits. Preferably, during the transformation step 400, the descriptors of the plurality of descriptors are each transformed into data coded on two bits.

Generally, all calculated descriptors will be subject to an encoding. However, it is possible that not all calculated descriptors are subject to an encoding. For example, the method according to the invention can include a step of selecting the descriptors that will or will not be subject to encoding.

Generally, this encoding will be exclusively associated with a descriptor. However, it is possible that several descriptors contribute to the definition of a value encoded on at most eight bits which will be complementary to encoded values specific to a descriptor. Furthermore, it is possible for a descriptor to be involved in the definition of multiple values encoded on at most eight bits. In one embodiment, during the transformation step 400, several descriptors could be used to generate a complementary two-bit coded data.

The inventors tested several methods for processing the data generated by the movement sensors. Furthermore, they tested several methods for reducing the dimensionality of the data produced by these movement sensors. However, it is the method according to the present invention that made it possible to achieve the best results in terms of energy consumption and data compression while retaining the useful information of the transmitted data to correctly represent the displacement of the moving entity such as the gait of the user.

Table 1 below represents normalized data of the system performances based on the processing of the data acquired from the data sensors before transmission.

TABLE 1

| | | Retention | |
|---|---|---|---|
| | Consumption | of the useful | Required |
| | of the | information of | storage of |
| | battery | the data | the data |
| No coding | 100 | 1 | 100 |
| Coding on one bit; | 1 | 38.2 | 1 |
| 4 descriptors | | | |
| Coding on two bits; | 5.5 | 23.7 | 3.7 |
| 4 descriptors | | | |
| Coding on three bits; | 15.1 | 14.2 | 5.2 |
| 4 descriptors | | | |
| Coding on four bits; | 21.9 | 9 | 16 |
| 4 descriptors | | | |
| Coding on one bit; | 3.1 | 24.1 | 3.2 |
| 8 descriptors | | | |
| Coding on two bits; | 10.8 | 11.9 | 7.3 |
| 8 descriptors | | | |
| Coding on three bits; | 26.5 | 8 | 16.5 |
| 8 descriptors | | | |
| Coding on four bits; | 34 | 4.9 | 29.1 |
| 8 descriptors | | | |
| Coding on eight bits; | 59 | 2.4 | 42.6 |
| 8 descriptors | | | |

Thus, the battery consumption values correspond to battery consumption values normalized based on the highest consumption (100) and on the lowest consumption (1). The retention values of the useful information of the data correspond to normalized values based on an ability to retain a maximum amount of information (1) and on a minimum retention (100). Particularly, the retention of the information of the data is estimated through the accuracy of the predictions of the movements by a set of machine learning and supervised models based on the encoded data. Finally, the required storage values of the data correspond to the volume in terms of normalized memory space from the highest value (100) to the lowest value (1).

Preferably, during the transformation step 400, at least eight descriptors are used and each coded on at least one bit, more preferably at least two bits. Generally, during the transformation step 400, descriptors are each coded on at most eight bits, preferably at most six bits and even more preferably at most four bits. Preferably, the encoding is a digital encoding unlike the encoding of an output voltage from an inertial unit which is generally derived from an analog processing.

For example, among the preferred embodiments, during the transformation step 400, the coding is carried out on:
one bit for between 4 and 20 descriptors, for example four descriptors or eight descriptors;
two bits for between 4 and 20 descriptors, for example four descriptors or eight descriptors;
three bits for between 4 and 20 descriptors, for example four descriptors.

The coding will be preferably carried out for each of the time windows 31.

Thus, the data encoded on at most two bits and associated with a time window 31 will be preferably representative of what could be considered as a phoneme of movement of the moving entity or of the arm of the user equipped with a watch or a bracelet or of the shoe. Alternatively, these data encoded on at most two bits and associated with a time window 31 can be considered as a phoneme if a phonemization of the movements associated with the displacement of the moving entity particularly with the gait is considered.

Advantageously, several encoded data (e.g. a plurality of descriptors each encoded on at most two bits) characterize a movement phoneme and several movement phonemes characterize a movement.

Thus, the data encoded under a series of values encoded on at most two bits associated with a time window 31 are representative of a micromovement of the user, particularly of an object such as a watch or a bracelet or a shoe worn by the user and more particularly of a sole 13, 14.

The table 2 below illustrates an example of data obtained following a transformation step according to the invention.

TABLE 2

| | FT1 | | FT2 | | FT3 | |
|---|---|---|---|---|---|---|
| | Calcu- | En- | Calcu- | En- | Calcu- | En- |
| | lated | coded | lated | coded | lated | coded |
| Descriptor 1 | 12.45989 | −1 | 70.8759 | 1 | 49.3476 | 0 |
| Descriptor 2 | 9.7228 | −1 | 23.7459 | 0 | 93.8016 | 2 |
| Descriptor 3 | 69.7904 | 1 | 69.0302 | 1 | 1.6486 | −1 |
| Descriptor 4 | 17.9573 | −1 | 24.2259 | 0 | 38.1186 | 0 |
| Descriptor 5 | 24.2259 | 0 | 147.2232 | 2 | 72.8551 | 1 |

The coding of the descriptors and therefore of the values obtained can be carried out on the basis of predetermined intervals. For example, within the framework of the descriptor 1 which can correspond to a maximum acceleration value on the axis Y, the method according to the invention can involve predetermined rules according to which a value less than 20 will be transformed into a value “−1”, a value from 20 to 50 (50 inclusive) will be transformed into a value “0”, a value comprised between 50 (50 exclusive) and 80 (80 exclusive) will be transformed into a value “1”, and a value greater than or equal to 80 will be transformed into a value “2”. Thus, the descriptor 1 is transformed into a data coded on at most eight bits, in this case 2 bits.

The predetermined intervals can be modified over time either automatically based on the obtained values or through updates.

The coding of the descriptors can also correspond to comparisons of values of different descriptors or of the same descriptor over several time windows 31. For example, if the descriptor 3, which can correspond to the average of the acceleration on the axis X, does not vary by more than 10% between the windows FT0 and FT1 then its value will be transformed into a value “1”. In the same way, the descriptor 3 does not vary by more than 10% between the windows FT1 and FT2 then its value will be transformed into a value “1”. On the other hand, the descriptor 3 varies by more than 80% downward between the windows FT2 and FT3 then its value will be transformed into a value “−1”.

Thus, the coding of the descriptors, that is to say the generation for each of the descriptors of a data coded on at most eight bits, can be advantageously based on predetermined rules. Furthermore, it can advantageously take into account predetermined intervals, evolutions of values over time and/or results of mathematical operations between the descriptors.

A method according to the invention could lead, as will be described subsequently, to the generation of a data tensor that, according to the example above, could be represented in the following form:

$$V 1 = \{ - 1 , - 1 , 1 , - 1 , 0 ; 1 , 0 , 1 , 0 , 2 ; 0 ; 2 , - 1 , 0 , 1 \}$$

Such a data tensor includes several sets of data encoded in at most two bits. For example, it includes a set of five values encoded in at most two bits corresponding to the encoding of descriptors for the time window denoted “FT1”; a set of five values encoded in at most two bits corresponding to the encoding of descriptors for the time window denoted “FT2”; and a set of five values encoded in at most two bits corresponding to the encoding of descriptors for the time window denoted “FT3”.

Thus, if the time windows FT1, FT2 and FT3 (table 2) correspond to a time sequence 32 and therefore to a movement unit then said movement of one second can be described by fifteen values encoded on two bits, i.e. in the case illustrated by 30 bits. Furthermore, in the case of using a sliding time sequence 32, such a movement could be represented by five time windows 32, i.e. 150 bits.

This is in comparison with the 14,400 bits of the data from movement sensors such as 9-axis inertial units sampling a one-second movement at 100 Hz.

The present invention therefore allows, with a minimal reduction in the information, a reduction in the necessary data streams of an order of about one hundred, leading to a reduction in energy consumption and in the necessary storage capacities.

Thus, a method according to the invention can also include a step 500 of generating a data tensor including the coded data obtained for each of the time windows 31.

The data tensor including the coded data obtained for each of the time windows 31 denoted “FT” in table 2 can correspond to a plurality of data tensors each including the coded data obtained for a time window 31.

Thus, the data tensor preferably includes an encoding of a combination of several movement phonemes of the moving entity and particularly of the user preferably of a watch or a bracelet or a shoe and particularly a sole. The data tensor advantageously includes an encoding which is representative of a position or movement of the watch or the bracelet worn by the user such as a shoe worn by a user.

A data tensor according to the invention advantageously makes it possible to identify a physical state and particularly a movement of a moving entity. A movement can correspond to a succession of positions. If all positions are the same then there is no movement. If at least one position is different then there has been a movement.

The inventors have further shown that the use of such a data tensor, including data coded on at most eight bits, preferably at most three bits, more preferably at most two bits, makes it possible to improve the performance of a machine learning model based on such a data tensor. Indeed, a machine learning model based on an encoding according to the invention will present, where appropriate, a reduced neural network size and/or a reduced calculation time while being tolerant to the statistical variabilities of the populations representative of the sets of data and flexible to the mechanical sensitivity of the inertial sensors.

Advantageously, the data tensor includes a series coded on at most four bits, preferably at most three bits and more preferably at most two bits. However, it could include other data which will not be necessarily coded on at most two bits. For example, the data tensor could include unencoded data that could correspond to attributes calculated for example at the level of the watch or the bracelet or the sole and describing an aspect of the moving entity (e.g. the user) and particularly of the user's gait.

Alternatively, the data tensor 34 corresponds to a series coded on at most eight bits, preferably at most three bits, preferably at most two bits.

The generation step 500 can include the generation of several data tensors for a time window 31. This is for example the case if a data tensor is generated for each of the axes of an inertial platform. Alternatively, a data tensor can include the coded data concerning several axes of an inertial platform.

A method according to the invention can also include a step 600 of transmitting the data tensor.

Advantageously, the data being coded on at most eight bits, the transmission can advantageously be the subject of concatenation within data frames allowing the transfer of data frames for example of 8 bits, 16 bits or 32 bits. Thus the number of messages is reduced by the optimization of the frame occupancy. Thus the transmission frames are optimized reducing the energy cost and the packet verification cost (e.g. Ack . . . ). This is particularly significant within the framework of the low energy transmissions such as BLE (Bluetooth Low Energy) or Zigbee.

Although a step of determining a movement or more generally a displacement of the moving entity (e.g. the user) of a user's gait can be carried out at the level of the device carrying out the step 200 of acquiring data generated by the movement sensor (s), it is also possible to divide the processing operations and to interpret the data tensor in an electronic device different from the electronic device that generated the data tensor.

The transmission could for example be carried out by wireless communication means such as radio communications. The transmission of the data tensor is then preferably done to an external mobile device. The transmission of the data tensor could also be done to a remote computing device such as a data processing server.

The transmission of the data tensor can also be done directly to one device for processing the data measured by one movement sensor to another, for example from a watch to a bracelet or from a bracelet to another bracelet, also from one sole to another sole of the same pair of shoes. The transmission of the data tensor can be carried out continuously with the transmission of one data tensor at a time. It can also correspond to the transmission of several data tensors. For example, a transmission can include several data tensors each corresponding to a different time window or to data tensors generated from different movement sensor variables.

According to another aspect, the present invention relates to a method 2 for determining a displacement of moving entity. Preferably, the method 2 for determining a displacement of a moving entity corresponds to a method for determining the gait of a living being, preferably a human being, using the encoding device 20.

Preferably, the determination of the displacement of the moving entity or the determination of the gait corresponds to the determination of a position, a movement, or an activity of the moving entity, or particularly to a position, a movement or an activity of the user.

Particularly, the present invention relates to a method 2 for determining the gait of a user. Preferably, the user's gait corresponds to a posture, movement or activity of the user.

The determination method 2 will be preferably executed by one or several processors. Advantageously, the method 2 for determining a displacement of a moving entity (e.g. a user) such that the determination 2 of the gait of a user is implemented at least partly at the level of an encoding device 20, preferably coupled to the user, preferably positioned on an arm of the user or in a shoe, for example in a watch, a sole, at the level of a shoe or in a bracelet worn by the user. Alternatively, the determination method 2 is implemented at least partly at the level of an external terminal 30 such as a mobile device, or a computing infrastructure.

Such a determination will be preferably made from encoded data, or capable of being encoded, according to an encoding method 1 according to the present invention.

Particularly, the method 2 for determining a displacement of a moving entity (e.g. a user) such that the determination 2 of the gait according to the invention will be based at least partly on encoded data. Particularly, the encoded data take the form of a time series for which each time window 31 includes at least four data encoded on at most eight bits, preferably at most three bits, more preferably at most two bits. Preferably, each time window 31 includes between 4 and 50 data encoded on at most eight bits, preferably at most three bits, more preferably at most two bits. The time series can correspond for example to one or several movement unit (s) and each of the movement units being formed by several time windows 31.

Particularly, the method 2 for determining a displacement of a moving entity (e.g. a user) such that the determination 2 of the gait according to the invention will be based at least partly on encoded data which take the form of one or several data tensors including for each time window 31 at least four data encoded on at most eight bits, preferably at most three bits, more preferably at most two bits. Preferably, each time window 31 is represented by 4 to 50 data encoded on at most three bits, preferably at most two bits. The one or several data tensors correspond for example to one or several movement (s) and each of the movements being formed represented by several time windows.

As illustrated in FIG. 4, the method 2 for determining a displacement of a moving entity (e.g. a user) such that the determination 2 of the gait includes: a step 710 of loading one, preferably several data tensors; and a step 720 of processing the data tensors preferably by a machine learning model trained so as to identify a position or movement.

The method 2 for determining a displacement of a moving entity (e.g. a user) such as the determination 2 of the gait preferably corresponds to a real-time determination of one or several elements constituting the displacement of a moving entity, e.g. of a user such as the gait of a user. That is to say, after the generation of data by one or several movement sensors, preferably in less than 60 seconds, more preferably in less than 30 seconds, even more preferably in less than 10 seconds, the method according to the invention is able to determine an element of the displacement of a moving entity (e.g. a user) like the user's gait such as a position, a posture, a step, a jump, or any other movement constituting the displacement of the moving entity (e.g. the gait of a user).

As illustrated in FIG. 4, the determination method 2 according to the invention can also include the following steps: a step 730 of selecting a position, posture or movement for a time interval from the positions, postures or movements identified for contiguous time sequences, a step 740 of comparing the position, posture and/or movement identified from data from different sensors and a step 750 of transmitting data to an external terminal.

As mentioned, the method 2 for determining a displacement of a moving entity (e.g. determining the gait of a user) includes a step 710 of loading one or several data tensors.

The loading of the data tensor can correspond to the loading of multiple data tensors. This step can correspond particularly within the framework of the invention to storing, preferably in a random access memory, one or several data tensors. This acquisition can also correspond to a loading into the memory of one or several processors intended to process them.

As mentioned, the determination method 2 includes a step 720 of processing the data tensor (s) by a machine learning model.

Particularly, the step 720 of processing the data tensor by a machine learning model will include the identification of classes of events from the data tensor (s). Particularly, the processing step 720 makes it possible, from several data tensors, preferably each corresponding to a movement phoneme, to identify a class of events. Preferably, the processing step 720 is carried out on third-order data tensors including several data tensors.

FIG. 3C illustrates for example three data tensors 34 in the form of three second-order tensors each including five data tensors. Each data tensor here corresponds to data calculated from a time window 31 and a data tensor 34 can then correspond to a time sequence 32. A second-order tensor illustrated in FIG. 3C includes data coded on one bit (0 or 1) for seven descriptors and over five time windows 31.

In FIG. 3C, the tensors 34 correspond to sliding and overlapping time windows 32. A time sequence 32 could also correspond to a window whose size varies with the calculations of the position, posture, movement or more broadly class of event. For example, a first time sequence 32 includes three time windows 31, a second time sequence 32 includes four time windows 31 and so on until reaching a predetermined value of maximum dimension of time sequence 32. The size of the time sequence 32 could then be reduced to a predetermined value of minimum dimension.

Thus, the data coded under bits associated with one or several time windows 31, preferably representative of a movement phoneme, will be processed by a machine learning model to infer a class of event such as a class of movement, position or posture. A class of movement can correspond within the meaning of the invention to a movement taking place over a very short duration, for example less than 500 ms and can correspond to portions of longer movement. For example, it will be possible within the framework of the invention, from the bits associated with a time window 31 or with a time sequence 32, to determine whether there is a current kneeling, a forward step, a backward step, etc.

Advantageously, the solution according to the invention uses one algorithms derived from machine, or several preferably previously calibrated, learning for the processing of the data tensor(s). These algorithms may have been built from different supervised, unsupervised learning models, or by reinforcement. An unsupervised learning algorithm can for example be selected from an unsupervised Gaussian mixture model, an ascending hierarchical classification (Hierarchical clustering Agglomerative), a descending hierarchical classification (Hierarchical clustering divisive). Alternatively, the algorithm is based on a supervised statistical learning model configured to minimize a risk of the scheduling rule and thus making it possible to obtain more efficient prediction rules. In this case, the determination and estimation calculation steps can be based on a model, trained on a set of data and configured to predict a label (e.g. displacement/gait similar or not to the displacement/gait recorded). For example, for calibration purposes, it is possible to use a set of data representative of a situation whose label is known. The set of data can also comprise multiple labels. The algorithm can be derived from the use of a supervised statistical learning model selected for example from the kernel methods (e.g. Support Vector Machines SVM, kernel Ridge regression), ensemble methods (e.g. decision trees, random forests), hierarchical partitioning, k-means partitioning, logic regression or neural networks.

Preferably, the trained machine learning model is preferably selected from a neural network, for example a deep learning model, a recurrent neural network (e.g. an LSTM for Long Short-Term Memory), a multilayer perception or other machine learning methods such as methods based on decision trees (e.g. random forest) or clustering methods (e.g. partitioning method or hierarchical methods).

Furthermore, advantageously, the method according to the invention can include a step 730 of selecting a position, posture or movement from the postures or movements identified for contiguous time sequences 32. This position, posture or this selected movement can particularly correspond to the position, posture or movement of a user during a particular time interval 33 having been the subject of several contiguous time sequences 32 as is illustrated in FIG. 3B. The time interval 33 can correspond to a movement unit.

Thus, the step 730 of selecting a position, posture or movement can be considered as a validation of a movement, position or posture based on the processing of several classes of movement, position or posture inferred during the step 720 of processing the data tensor by a machine learning model.

The selection step 730 could include a comparison of the position, posture or movement identified for contiguous time sequences 32. Indeed, particularly when a sliding window is used, or more generally when a movement is represented by several contiguous time sequences, as it is the case in FIG. 3B, the method according to the invention can include an identification of a position, posture or movement for each of the several contiguous time sequences 32. Then, the method includes a selection of the position, posture or movement most represented within the contiguous time sequences 32 studied. This reduces the risk of false positives by taking advantage of the real-time recognition of position, posture or movement within time series and of a multiple validation of the same position, posture or movement. For example, a position, posture or movement can be selected from an analysis of at least three, preferably at least four and even more preferably at least five contiguous time sequences.

Preferably, the selection step 730 is carried out on third-order data tensors including several data tensors.

Furthermore, advantageously, the method according to the invention could also include a step 740 of comparing identified positions, postures and/or movements, for example for a given instant, from data from different sensors.

Such a comparison 740 of identified positions, postures and/or movements can be used to determine the displacement of the moving entity, e.g. the user's gait by the association of information coming from several sensors.

This comparison 740 is preferably made for synchronized time intervals so that the measured and interpreted data correspond to the same moment of the displacement of the moving entity, e.g. the user's gait. Such synchronization is possible for example through the use of a synchronized clock or inter-device communications.

Particularly, the determination method 2 can be implemented from data tensors coming from several sources. For example, several movement sensors positioned at different points of a moving entity (e.g. of a user) also generating data tensors according to the present invention.

Under these conditions, the determination method 2 can advantageously include, for a given time interval, a comparison 740 of the position, posture, and/or movement, identified based on the data from a first sensor with the position, posture, or movement, identified from a second sensor. For example, the step 740 of comparing the identified position, posture, and/or movement can include a comparison of the position, posture, or movement identified from a sensor positioned on the right arm of a user with the position or movement identified from a sensor positioned on the left arm of the user. Particularly, the step 740 of comparing the identified posture and/or movement can include a comparison of the posture or movement identified from a sensor positioned on the right foot with the posture or movement identified from a sensor positioned on the left foot.

The step 740 of comparing positions, postures and/or movements identified for a given instant for different sensors could implement positions, postures or movements identified from data from different sensors not implementing the data tensor according to the invention. For example, the position, posture or movement, identified from encoded data according to the invention could be compared with the position, posture or movement, identified with other methods.

Particularly, the determination method 2 can advantageously include a comparison of the position, posture or movement identified for each of the arms and/or feet of a user with a reference frame. Such a comparison will make it possible to reinforce the specificity of the determination performance and in some cases to determine a type of movement or a complex posture. Indeed, some movements or postures could be determined accurately only by taking into account the results of several movement sensors. The reference frame could consist of a database, an algorithm transcribing expert rules, or a machine learning model.

Preferably, the method can also include a step 750 of transmitting data to an external terminal 30. This transmission is preferably carried out occasionally. Particularly, this can correspond to the transmission of all the data generated and/or calculated to an external terminal 30.

Furthermore, the method can include the transmission of the data received by the external terminal 30 to one or other external terminal (s) that could be involved in the calculation of other parameters of the displacement of the moving entity (e.g. of the gait of the user) or in their display.

The transmitted data can for example be raw data as generated by the movement sensors, preprocessed data, transformed data or displacement data (e.g. gait data).

Furthermore, a determination method 2 according to the invention can also include a step 800 of identifying an activity associated with one or several identified movements, positions and/or postures.

This identification of an activity preferably includes the comparison of activity reference values with the values of one or several data tensors or with the values of one or several identified movements, positions or postures.

It is then possible to determine, preferably from several identified movements and/or positions, on the one hand what is the type of moving entity whose movements/positions have been identified and on the other hand what activity is practiced by the moving entity. Indeed, those skilled in the art will understand that depending on the type of: moving entity considered, the movements and positions identified will be radically different and will make it possible to characterize the moving entity. Furthermore, when the moving entity is for example a user, it will be possible to determine the type of activity practiced by the latter on based the movements/positions identified. It is then possible to determine, preferably from several identified movements and/or postures, what activity is practiced by the user.

Below, the different mandatory or non-mandatory steps of a method 2 for determining a displacement of a moving entity (e.g. the gait of a user) are presented to summarize and put the present invention into perspective.

During the processing 720 of the data tensor by a machine learning model, the present invention could for example study a plurality of time windows 31 so as to identify a class of movement, for each of the time windows 31.

More preferably, during the processing 720 of the data tensor, particularly of a third-order data tensor, by a machine learning model, the present invention could study a plurality of time windows 31 so as to identify a class of movement, such as a step, for each of the time sequences 32 including several time windows 31.

An optional complementary step 730 of selecting a posture or movement can be implemented so as to confirm, by analyzing the classes of movement identified for several contiguous time windows 31 or for several time sequences 32, the presence of a particular movement such as flexion, translation, rotation, extension, step. For example, a time sequence 32 can indicate a step while the next three indicate a slip. In this case, at the end of the complementary step 730 of selecting a posture or movement, the present invention could indicate the occurrence of a slip.

Furthermore, more generally, the analysis of the classes of movement identified for several contiguous time windows 31 or for several time sequences 32 makes it possible to indicate the occurrence of a “risky” movement. Indeed, when the identified movement is not associated with any class of inferred movement or posture, it can be the consequence of an involuntary movement of the user and potentially a source of muscular and/or skeletal injury for the user. This can be particularly applicable in physically demanding jobs that can lead to wounds, in particular to the lower limbs, such as building and public work trades.

In an optional complementary step 740 of comparing identified postures and/or movements, the present invention will study data generated by other sensors, preferably also in the form of a data tensor (e.g. a third-order data tensor), for example a data tensor generated from the movements of the left foot. In the remainder of the example above, the analysis of the data tensor (s) coming from the left foot can indicate a flexion of the knee. In this case, the movement identified at the end of step 740 could for example be representative of a running movement of the user or a protection movement in boxing or a semicircular gait. Thus, the movement or posture identified at the end of step 740 can be considered as a more complex movement. Indeed, the data tensors, more particularly the level-3 tensors, can be advantageously associated with a particular displacement pattern describing one or several specific movements.

Finally, in a step 800 of identifying the activity, the present invention could, from generated data, determine several movements that will for example lead to identifying that the activity carried out is a sports activity of the judo, running or boxing type.

Preferably, the method can also include a data storage step 900. This memorization is preferably done continuously. Particularly, this can correspond to the storage of all the data generated and/or calculated within the framework of a method according to the invention. The stored data can for example be raw data as generated by the movement sensors, preprocessed data, transformed data or displacement data (e.g. gait). Preferably, the stored data correspond to transformed data or displacement data (e.g. gait).

Advantageously, as opposed to the raw and/or preprocessed data which are saved over a short duration (e.g. less than 5 minutes) for example on a cache memory, a data tensor or displacement value (e.g. gait) is stored in the longer term, for example on a memory.

Advantageously, the calculation of a determined data tensor and/or a displacement (e.g. of a gait) is done in real time, that is to say less than 1 hour after the generation of the data by the one or several of the movement sensors coupled to the moving entity (e.g. a sole), preferably less than 10 minutes, more preferably less than a minute, even more preferably less than 10 seconds.

According to another aspect, the invention relates to a device 20 for encoding data generated by one or several movement sensors 21, preferably coupled to a moving entity, for example to a watch or a bracelet or a shoe worn by a user. Such a data encoding device 20 can be particularly used to identify a position or movement of a moving entity, e.g. of a user on a time sequence 32, in particular when the shoe is worn by a user. More broadly, if it is applied to movement sensors 21 positioned on the body of a user, it can be used to identify a position or movement of the user. More specifically, such an encoding device 20 is particularly advantageous when it is used to identify a movement of a user when he/she wears a watch 11 or a bracelet 12. For example, such an encoding device 20 is particularly advantageous when it is used to identify a position or movement of a shoe sole 13, 14 when inserted into a shoe or integrated to a shoe.

This encoding device 20, weighing only a few grams and being of reduced dimension, can be housed in a space-saving manner in any accessory, for example an insole and/or outsole. This low volume limits the impact on the comfort of the user and has the advantage of optimizing the production costs by making it less expensive and simpler to integrate this technology into any type of accessory worn by the user or directly in the moving entity during the industrial process.

An encoding device 20 according to the invention is detailed in FIG. 5. The different components of the encoding device 20 are preferably arranged on an electronic card 27 (or printed circuit), but the invention can provide various types of arrangement, for example a single module combining all of the functions described here. Likewise, these means can be divided into several electronic cards or brought together on a single electronic card. Furthermore, when an action is attributed to a device, a means or a module, this is in fact generally performed by a microprocessor of the device or module controlled by instruction codes recorded in a memory. Likewise, if an action is attributed to an application, it is in fact performed by a microprocessor of the device in a memory in which the instruction codes corresponding to the application are recorded. When a device or module emits or receives a message, this message is emitted or received by a communication interface.

Such an encoding device 20 according to the invention can include a movement sensor 21 such as an inertial platform configured to generate data on the displacement of a moving entity such as the gait of a user. During the use of the encoding device 20 by a user (the encoding device is advantageously positioned on a user), the inertial platform acquires signals representative of a movement parameter (acceleration and/or speed, for example angular speed) of the user, preferably of the user's foot, along the axes X, Y, Z. Furthermore, these data can then be processed to generate at least one acceleration signal. The inertial platform is for example made up of at least an accelerometer and a gyroscope. Preferably, it includes several accelerometers and gyroscopes. The encoding device 20 can also include one or several magnetometers so as to acquire three additional raw signals corresponding to the magnetic field values in three dimensions.

Each encoding device 20 can also include other sensors, in particular an inclinometer, a barometer, a temperature sensor and an altimeter to benefit from increased accuracy.

Preferably, an encoding device 20 includes one or several processors 22 configured to execute one of the methods according to the invention, preferably the encoding method, and their preferred, advantageous or non-advantageous different embodiments.

Particularly, the one or several processors 22 are configured so as to acquire data generated by the movement sensor (s) 21, said data including acceleration and angular speed values.

The one or several processors 22 can also be configured so as to calculate a plurality of descriptors from the data acquired over a time window 31, the calculation step being repeated for several consecutive time windows 31, the consecutive time windows 31 forming a time sequence 32.

Furthermore, the one or several processors 22 can also be configured so as to transform the plurality of calculated descriptors, said step including a coding of the descriptors each on at most two bits, said bits associated with a time window 31 being representative of a movement phoneme.

Also, the one or several processors 22 can be configured so as to generate a data tensor including the bits obtained for each of the time windows 31, so that the data tensor includes an encoding of a combination of several movement phonemes which is representative of a posture or movement of the user.

Preferably, the one or several processors 22 will be configured so as to:
acquire data generated by the movement sensor (s) 21,
calculate a plurality of descriptors from the data acquired over a time window 31, the calculation step being repeated for several consecutive time windows 31; and
transform the plurality of calculated descriptors, said step including a coding of the descriptors each on at most two bits, said bits associated with a time window 31 being representative of a movement phoneme.

Furthermore, the one or several processors 22 will be configured so as to generate a data tensor including the bits obtained for each of the time windows 31, so that the data tensor includes an encoding, preferably of a combination of several movement phonemes, which is representative of a position, posture or movement of the moving entity (e.g. the user).

Particularly, the one or several processors 22 can be configured so as to execute the following actions:
loading data coded on at most eight bits associated with the time sequence 32, being representative of a movement of a moving entity on said time sequence 32, for example in the form of one or several data tensors; and
processing the data tensor (s) by a machine learning model trained to identify a position or movement of the moving entity.

Also, the one or several processors 22 can be configured so as to execute the following actions: loading data coded on at most eight bits associated with the time sequence 32, being representative of a position or movement of the shoe on said time sequence 32, for example in the form of one or several data tensors; and processing the data tensor (s) by a trained machine learning model so as to identify a posture or movement of the user.

Alternatively, the processor (s) can be configured to generate the data tensor then send it to an external terminal 30 so that the displacement of the moving entity (e.g. the gait of a user) can be determined.

Thus, the encoding device 20 according to the invention advantageously includes one or several communication means 25. The one or several communication means 25 are able to receive and transmit the data on at least one communication network. Preferably, the communication is carried out via a wireless protocol as Wi-Fi, 3G, 4G, 5G and/or Bluetooth. Preferably the communication protocol is a BLE or ANT+ protocol. These communications protocols allow low energy consumption.

Preferably, the encoding device 20 according to the invention includes a first communication means 25 configured so that the encoding device 20 is able to transmit at least part of the data tensor to an external terminal 30. These data can be transmitted in real or delayed time to an external terminal 30. The external terminal 30 can for example be a remote system such as a tablet, a mobile telephone (Smartphone), a computer or a server. As has been mentioned, the encoding device 20 according to the invention can also implement a step of determining the displacement of the moving entity, such as the gait of a user. Thus, it can be configured to send posture or movement data identified for a user. These data can be transmitted in real or delayed time to an external terminal 30.

Advantageously, an encoding device 20 according to the invention further includes a second communication means configured so that a first encoding device 20 is able to communicate with a second encoding device 20.

Particularly, the encoding devices 20 can be configured to communicate with each other and to initiate the generation of data on the movement of a user only after receiving a message from the other encoding device 20. This contributes to the synchronization of the encoding devices 20. For example, the encoding devices 20 can be configured to associate and/or match timestamps of the samples derived from the inertial platforms.

Advantageously, if an encoding device 20 were to disconnect or lose the synchronization over time relative to the other encoding device 20, a method according to the invention could comprise a step of synchronizing the encoding devices 20. Thus, a search signal is sent by the connected encoding device 20, the disconnected encoding device 20 receives the search signal and synchronizes on the connected encoding device 20.

The encoding device 20 according to the invention also includes a data memory 23 configured to store at least part of the acquired data and/or data tensors. The encoding device 20 according to the invention is such that it can operate with a low-capacity data memory 23. It can be configured to store the raw data generated by the inertial platform. Advantageously, the data memory 23 is configured to store at least part of the transformed data, but not to store the generated raw data. Thus, its capacity is not burdened by generated raw data. The transformed data can correspond to data preprocessed by the processor (s) or to data tensors.

Furthermore, the encoding device 20 according to the invention can include an energy source 26. The energy source is preferably of the rechargeable or non-rechargeable battery type. Preferably the energy source is a rechargeable battery. Furthermore, it can be associated with a system for charging through the movement or through external energy. The external energy charging system can in particular be a wired connection charging system, an induction or a photovoltaic charging system.

The energy source 26 is preferably of the rechargeable or non-rechargeable battery type. Preferably the energy source is a rechargeable battery. The recharging can be carried out using different technologies such as:
by charger, with a connector flush with the watch, the bracelet or the sole;
with a mechanical charging device integrated to the watch, the bracelet or the sole, for example a piezoelectric device able to provide electrical energy from walking;
with a contactless device, for example by induction; and/or
with a photovoltaic device.

Furthermore, the encoding device according to the invention can include a port for wired connection 28, preferably protected by a removable tab. This port for wired connection can for example be a USB or firewire port. Advantageously, the USB port is also resistant to water or humidity. Furthermore, the USB port is advantageously topped with a polymer beam to give it greater resistance in use condition. This port for wired connection 28 can be used as mentioned above to recharge the battery but also to exchange data and for example update the firmware of the electronic card carrying the different components of the encoding device. Preferably, the removable tab or USB cover protects the USB port from foreign bodies. For example, the removable tab makes it possible to protect the USB port from water or dust. Such a tab can preferably be made of elastomer or polyurethane type polymer.

Furthermore, according to another aspect, the present invention relates to a system 5 for determining a displacement of a moving entity, particularly the gait of a user.

Such a determination system 5, described in connection with FIGS. 6A and 6B, will advantageously include one or several encoding devices 20 according to the present invention. Particularly, the determination system 5 according to the invention includes at least two encoding devices 20 according to the present invention.

Particularly, each of the encoding devices 20 is designed so as to be able to communicate independently with the other and/or directly with an external terminal 30 in order to be able to exchange own its information on the posture/movement/activity of the user and particularly his/her foot.

As shown in FIG. 6A, the system 5 according to the invention can include a watch 11 and a bracelet 12 or two bracelets 12, each including an encoding device 20 according to the invention. Conventionally, the watch 11 and bracelet 12 each include an encoding device 20 according to the invention. As shown in FIG. 6B, the system 5 according to the invention can include a pair of soles 13, 14 each including an encoding device 20 according to the invention. The soles 13, 14 usable within the framework of the system 5 according to the invention can for example correspond to outsoles or insoles of shoes. These soles can be removable or permanently integrated to the sole assembly of the shoes. Conventionally, the soles 13, 14 composing said pair of soles, each include an encoding device 20 according to the invention. As shown in FIG. 6B, the encoding device 20 is preferably positioned at a median portion of the sole.

As mentioned, the system 5 according to the invention can include an external terminal 30 configured to receive data coming from the encoding device (s) 20.

Advantageously, a dedicated application is installed on this external terminal in order to process the information transmitted by the encoding device (s) 20 and allow the user to interact with the invention. It is then possible, for example, to access this remote server via a web interface. All communications with the remote server can be secured for example HTTPS Transfer by (HyperText Protocol Secure) protocols and AES (Advanced Encryption Standard) encryption 512. Thus, this can allow, via a client, access to data by medical personnel in charge of monitoring the user.

The external terminal 30 is generally a tablet, a mobile phone (Smartphone), a gateway, a router, a computer or a server. It may be able to transfer these data to a remote server. It is then possible for example to access this remote server via a web interface. It could for example be configured to inference specific implement solutions to given applications from coded data, or coded data tensors or particularly order data tensors generated by the encoding device 20. It can also be configured to generate data on the displacement/gait from data derived from the step 700 of determining a displacement of the moving entity and therefore for example from the user's gait.

Thus, the user can access data related to his/her displacement/gait such as data relating to his/her daily physical activities and relating to several biomechanical parameters, such as posture, pronation/supination, impact force, step length, contact time, lameness, balance and several other parameters relating to the user and describing his/her displacements, walking, postures and movements and thus to follow their evolution.

1. A method for encoding data generated by one or several movement sensors coupled to a moving entity, for the identification of a position or movement of the moving entity on a time sequence,
said method, executed by one or several processors, including:
a step of acquiring data generated by the movement sensor(s) coupled to the moving entity, said data including variable values in the form of time series;
a step of calculating, from the data acquired over a time window, a plurality of descriptors, the calculating step being repeated for several consecutive time windows, the consecutive time windows forming the time sequence; and
a step of transforming the plurality of descriptors of each of the consecutive time windows forming the time sequence,
said transforming step including a generation, for each of the descriptors, of first data coded on at most eight bits,
said first data coded on at most eight bits associated with the time sequence being representative of a position or movement of the moving entity on said time sequence.

2. The data encoding method according to claim 1, further comprising a step of generating a data tensor including the first data coded on at most eight bits obtained for several of the time windows, so that the data tensor includes an encoding representative of the position or movement of the moving entity.

3. The data encoding method according to claim 1, wherein the descriptors are calculated from mathematical functions applied to the preprocessed or non-preprocessed data generated by the movement sensor(s) coupled to the moving entity, and said applied mathematical functions including a calculation of average, of statistical asymmetry, of the Kurtosis coefficient, of comparisons of values of the extrema or quartiles, of positions of extrema in a time series, of comparisons of edge values of subsets of the movement and/or of a calculation of a data topology.

4. The data encoding method according to claim 1, wherein the time windows of two consecutive time sequences have different durations.

5. The data encoding method according to claim 1, wherein, during the transformation step, at least four descriptors are used to each generate a second data coded on at least one bit.

6. The data encoding method according to claim 1, wherein, during the transformation step, between four and twenty descriptors are used to each generate a third data coded on a number of bits selected from: one, two or three.

7. The data encoding method according to claim 2, further comprising a step of analyzing the first data coded on at most eight bits as a function of an activity data of the moving entity, said analyzing step comprising a comparison between the first data coded on at most eight bits and a database comprising reference data for the given activity.

8. The data encoding method according to claim 1, wherein, during the transforming step, one or several fourth data coded on at most eight bits are calculated from values of descriptors calculated from data acquired over consecutive time windows.

9. The data encoding method according to claim 1, wherein the moving entity is a shoe, and the acquiring step is carried out while the one or several movement sensors as well as the one or several processors are integrated in an encoding device.

10. A method for determining a displacement of a moving entity, from data encoded according to the encoding method according to claim 2, comprising:
a step of loading one or several said data tensors including an encoding representative of the position or movement of the moving entity, said one or several data tensors including the plurality of descriptors for each of the consecutive time windows forming the time sequence, each of the descriptors corresponding to a respective first data coded on at most eight bits; and
a step of processing the data tensor(s) by a machine learning model trained so as to identify a position or movement of the moving entity.

11. The method for determining a displacement of a moving entity according to claim 10, wherein the step of processing the data tensor(s) by a machine learning model includes identification of classes of events from the data tensor(s), namely one class of movement per time sequence.

12. The method for determining a displacement of a moving entity according to any one of claim 10, further comprising a step of selecting, by the trained machine learning model, a position and/or movement from a comparison of the positions and/or movements identified from contiguous time windows or contiguous time sequences.

13. The method for determining a displacement of a moving entity according to claim 10, further comprising a step of comparing the positions and/or movements identified for a given time window or time sequence from data from different sensors so as to identify the position and/or movement of the moving entity.

14. The method for determining a displacement of a moving entity according to claim 10, wherein the moving entity is a user of a shoe, the encoding representative of a position or movement of the moving entity is an encoding representative of a position or movement of the shoe of the user; and the step of processing the data tensor(s) makes it possible to identify a posture or movement of the user.

15. A device for encoding data generated by one or several movement sensors coupled to a moving entity, for identification of a position or movement of the moving entity on a time sequence, said encoding device including one or several processors, said one or several processors being configured to:
acquire the data generated by the movement sensor(s) coupled to the moving entity, said data including variable values in the form of time series;
calculate, from the data acquired over a time window, a plurality of descriptors, the calculation being repeated for several consecutive time windows, the consecutive time windows forming a time sequence;
transform the plurality of descriptors of each of the consecutive time windows forming the time sequence, said transformation including a generation for each of the descriptors of a data coded on at most eight bits, said data coded on at most eight bits associated with the time sequence being representative of a position or movement of the moving entity on said time sequence.

16. The device for encoding data generated by one or several movement sensors according to claim 15, wherein said one or several processors are configured to generate a data tensor including the data coded on at most eight bits obtained for several of the time windows, such that the data tensor includes an encoding representative of a position or movement of the moving entity.

17. The device for encoding data generated by one or several movement sensors according to claim 15, further comprising said one or several movement sensors.

18. The device for encoding data generated by one or several movement sensors according to claim 15, wherein it is integrated in a removable or non-removable sole.

19. The device for encoding data generated by one or several movement sensors according to claim 15, wherein said one or several movement sensors include an accelerometer and a gyroscope.

20. A system for determining a displacement of a moving entity, comprising the encoding device according to claim 15 and an external terminal configured to receive the generated data tensor and then to determine the displacement of a moving entity from the generated data tensor.
